SEQUENCE LISTING (3 sequences)

SEQ ID NO: 1 - DNA, 22 bp, synthetic construct
  source 1..22: mol_type = other DNA
agagggacatccaggactttgt

SEQ ID NO: 2 - DNA, 19 bp, synthetic construct
  source 1..19: mol_type = other DNA
caggcgcttgttggtgtac

SEQ ID NO: 3 - DNA, 19 bp, synthetic construct
  source 1..19: mol_type = other DNA
  modified_base 11: mod_base = OTHER; note = n = ZEN internal quencher from Integrated DNA Technologies
  modified_base 1: mod_base = OTHER; note = n= 5'-fluorescein phosphoramidite
  modified_base 19: mod_base = OTHER; note = n= 3' Iowa Black FQ from Integrated DNA Technologies
naccgccgaanctgagcan

(19) United States
(12) Patent Application Publication
(10) Pub. No.: US 2026/0263434 A1
(43) Pub. Date: Sep. 10, 2026

(54) INDOLINYL COMPOUND FOR TREATMENT OF VIRAL INFECTIONS UNRESPONSIVE TO TREATMENT WITH A FIRST ANTIVIRAL THERAPY

(71) Applicant: Gilead Sciences, Inc., Foster City, CA (US)

(72) Inventors: Alexandre MERCIER, Pacifica, CA (US); Varsha Varijakshan NAIR, Newark, CA (US); Roberto MATEO FERNANDEZ, San Francisco, CA (US); Lindsey A. MAY, San Francisco, CA (US); Pui Yan HO, Fremont, CA (US); Christopher M. RICHARDS, Foster City, CA (US)

(21) Appl. No.: 19/266,095

(22) Filed: Jul. 10, 2025

Related U.S. Application Data
(60) Provisional application No. 63/670,698, filed on Jul. 12, 2024.

(51) Int. Cl. A61K 31/4439; A61P 31/22

(52) U.S. Cl. CPC A61K 31/4439; A61P 31/22

(57) ABSTRACT

The present disclosure provides methods and pharmaceutical formulations for the treatment and prophylaxis of a viral infection unresponsive to treatment with one or more first antiviral therapy using an indolinyl compound.

### CROSS REFERENCE TO RELATED APPLICATION

This application claims the benefit of U.S. Provisional Application No. 63/670,698, filed Jul. 12, 2024, which is incorporated herein by reference in its entirety.

### SEQUENCE LISTING

The contents of the electronic sequence listing (434470_ST26.xml; Size 4,928 bytes; and Date of Creation May 19, 2026) is herein incorporated by reference in its entirety.

### BACKGROUND

Human herpes viruses are large-enveloped double-stranded DNA viruses that share the characteristic of establishing life-long infections in humans. This is accomplished by their ability to exist in the host either as a symptom free latent infection, where the virus lies dormant or, following activation, as a lytic infection with associated symptoms. These viral infections have widespread, worldwide prevalence and it is notable that over 90% of all humans are chronically infected with more than one human herpes virus.

Human herpes viruses are classified into three subfamilies (i.e., a, p and y) based upon their biological characteristics and the family consists of eight members, i.e., Herpes Simplex Virus subtype type 1 and 2 (HSV1, HSV2), Varicella Zoster Virus (VZV), Epstein-Barr virus (EBV), Cytomegalovirus (CMV), and human herpes viruses 6-8 (HHV 6-8).

HSV1 and 2 infections can cause disease in immune competent individuals. Both subtypes cause cutaneous genital/anal and orolabial/nasal cavity (cold sore) lesions, although HSV2 is more commonly associated with the former and HSV1 the latter such that >80% of genital infections are believed to be caused by HSV2. Globally, over 500 million people have genital herpes infections. Symptoms vary but are typically most severe on first time of infection and can last for weeks to months. Approximately 50 to 80% of the world's population have orolabial HSV infection, which is the main cause of cold sores. HSV, and particularly HSV1, can also cause lesions on the fingers (Whitlows) and other areas of the skin.

The vast majority of HSV infected individuals will not experience any noticeable symptoms. However, some will experience recurrent outbreaks of infection. In the USA, 20 to 40% of the population will get recurrent labial HSV lesions. Significantly, orolabial cold sores and Whitlow's provide a very easy route for transmission of the virus to other individuals which can lead to rarer but much more serious HSV-related pathologies. For example, HSV-related ocular keratitis is a major cause of blindness. HSV can also cause encephalitis in neonates which is a life-threatening condition. Other disorders also believed to be caused by HSV include herpes gladiatorum, Mollaret's meningitis and possibly Bell's palsy.

Primary infection with, or reactivation of an existing herpes virus infection, can be a major cause of disease in immunocompromised individuals. Key at-risk immunocompromised populations include patients undergoing solid organ or stem cell transplantation, individuals with HIV/AIDS, and ICU patients.

Presently, there is no cure for HSV. Medicines have been developed that can to some degree prevent or shorten outbreaks, but there is a need for improved therapies for treating HSV infection and inhibiting viral replication.

Currently, nucleoside analogues, such as acyclovir and its prodrugs, e.g., valacyclovir and famciclovir, are used as agents against herpes viruses such as HSV. In order to exert their effects, these nucleoside analogues must first be phosphorylated by viral thymidine kinase (TK) and then subsequently converted by cellular kinases to the nucleoside triphosphate, which inhibits the activity of the viral DNA polymerase. If the virus has no functionally active TK, as is the case, for example, with resistant HSV mutants or with TK-negative viruses, the active substance is unable to exert its effects.

Nucleoside analogues are clinically administered at a dose as high as several hundred in mg to several grams per day and even in high doses, and over long treatment durations, these compounds do not completely prevent recurrent outbreaks of symptoms from HSV infection. High doses also lead to increased levels of adverse effects.

Viral shedding is also common in HSV patients and can asymptomatically facilitate the transmission of HSV to more individuals. Nucleoside analogues do little to address this and long-term suppressive treatment, e.g., with valacyclovir has been shown to reduce transmission risk only by 46%. Since the nucleoside analogues can incorporate into the genome DNA of a host via the host DNA polymerase, the mutagenicity of these agents is also a concern, as documented for the nucleoside analogue, ganciclovir.

Given the inadequacy of existing treatments, there is an urgent medical need to develop improved, well-tolerated anti-herpes treatments.

A class of compounds being investigated for HSV treatment are the helicase-primase inhibitors. Helicase-primase inhibitors are antiviral agents with a novel mechanism of action against HSV1 and 2 (FIG. 1). They inhibit the viral heterotrimeric complex consisting of helicase, primase, and cofactor subunits that have functions essential for viral DNA replication. They are not nucleoside analogues and do not require phosphorylation by TK to inhibit HSV replication and they are therefore potentially active against TK-deficient HSV, which as described above, is a major mechanism of resistance to nucleoside analogues, such as acyclovir.

Two examples of helicase-primase inhibitors are BILS-179 BS and amenamevir (Katsumata et al. (2018) Biochem Pharm 158 p 201-206). BILS-179 BS has been dosed orally but was suspended from early clinical trials due to adverse events. Another example is pritelivir, a thiazolylamide derivative with the chemical name N-[5-(aminosulfonyl)-4-methyl-1,3-thiazol-2-yl]-N-methyl-2-[4-(2-pyridinyl)-phenyl]acetamide.

WO2000076966 discloses indolinylamide derivatives, a method for the production thereof, and the use of said compounds as medicaments, particularly as antiviral drugs. WO2018127207 discloses new thiazole compounds, pharmaceutical compositions thereof, and applications thereof in the preparation of drugs for the treatment of diseases related to herpes simplex viruses. WO2023225162 relates to indolinyl compounds that inhibit viral helicase-primase; the use of the compounds for the preparation a medicament for the treatment of diseases and/or condition through inhibiting viral helicase-primase; use of those compounds in the treatment of viral infections; and intermediates for its preparation and to pharmaceutical compositions containing those compounds.

There is still a need for additional antiviral compounds for the treatment and prophylaxis of HSV infections that are unresponsive to treatment with one or more first antiviral treatments and have an improved profile with respect to safety, potency, selectivity and/or bioavailability.

### SUMMARY OF THE INVENTION

One embodiment described herein is a method for the treatment of an HSV infection in a subject in need thereof, the method comprising: administering to the subject a therapeutically effective amount of Compound 1:

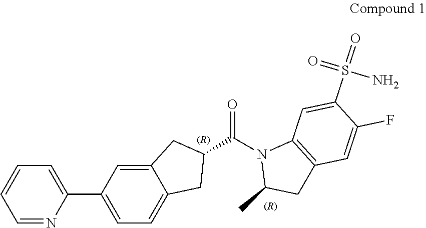

or a pharmaceutically acceptable salt thereof; wherein the virus is unresponsive to treatment with one or more first antiviral therapy. In one aspect, the first antiviral therapy is a nucleoside analogue or a helicase-primase inhibitor. In one aspect, the first antiviral therapy is a nucleoside analog, and the nucleoside analogue is acyclovir. In one aspect, the first antiviral therapy is a helicase-primase inhibitor, and the helicase-primase inhibitor is pritelivir.

In one aspect, the infection is a Herpes Simplex Virus (HSV) infection. In one aspect, the HSV is an HSV-1 or HSV-2. In one aspect, the HSV comprises one or more mutations. In one aspect, HSV comprises one or more mutations selected from the group consisting of K356N, K355N, K355T, K355R, L805I, S497N or S498N mutation in the UL5 gene. In one aspect, HSV comprises a A906V mutation in the UL52 gene. In one aspect, infection is resistant to treatment with one or more antiviral therapy.

In one aspect, Compound 1 is about greater than 1500 fold more potent than acyclovir. In one aspect, Compound 1 is about greater than 12 fold more potent than pritelivir.

Another embodiment described herein is a method for the treatment of a viral infection unresponsive to treatment with one or more first antiviral therapies in a subject in need thereof, the method comprising: administering to the subject a therapeutically effective amount of a pharmaceutical composition comprising Compound 1:

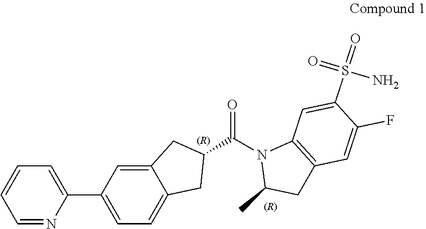

or a pharmaceutically acceptable salt thereof and a pharmaceutically acceptable excipient. In one aspect, the first antiviral therapy is a nucleoside analogue or a helicase-primase inhibitor. In one aspect, the nucleoside analogue is acyclovir. In one aspect, the helicase-primase inhibitor is pritelivir. In one aspect, the infection is an HSV infection. In one aspect, the HSV is an HSV-1 or HSV-2. In one aspect, the HSV comprises one or more mutations. In one aspect, the HSV comprises a K355 mutation in the UL5 gene. In one aspect, the HSV comprises a K356N mutation in the UL5 gene. In one aspect, the HSV comprises a S498N mutation in the UL5 gene. In one aspect, the infection is resistant to treatment with one or more antiviral therapy.

In one aspect, the Compound 1 is about greater than 1500 fold more potent than acyclovir. In one aspect, the Compound 1 is about greater than 12 fold more potent than pritelivir.

Another embodiment described herein is a method for the treatment of HSV infection previously treated with acyclovir, in a subject in need thereof, the method comprising: administering to the subject a therapeutically effective amount of Compound 1:

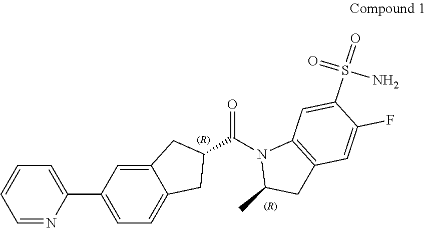

or a pharmaceutically acceptable salt thereof.

Another embodiment described herein is for the treatment of HSV infection previously treated with pritelivir, in a subject in need thereof, the method comprising: administering to the subject a therapeutically effective amount of Compound 1:

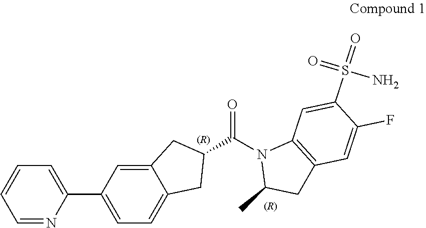

or a pharmaceutically acceptable salt thereof.

Another embodiment described herein is a method of treatment of an HSV infection previously treated with an antiviral therapy in a subject in need thereof, the method comprising: administering to the subject a therapeutically effective amount of Compound 1:

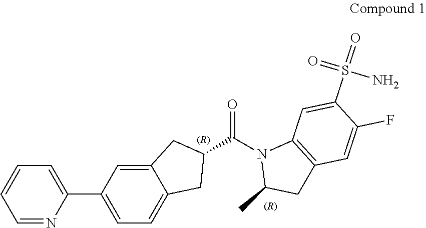

or a pharmaceutically acceptable salt thereof; wherein the HSV virus comprises one or more mutations. In one aspect the HSV comprises one or more mutations selected from the group consisting of K356N, K355N, K355T, K355R, L805I, S497N or S498N mutation in the UL5 gene. In another aspect, the HSV comprises a A906V mutation in the UL52 gene.

### BRIEF DESCRIPTION OF THE DRAWINGS

FIG. 1 describes data demonstrating Compound 1 is a potent Inhibitor of HSV-1 and HSV-2 laboratory strains. Data are mean±SD. ARPE-19, human retinal epithelial cells; EC₅₀, half-maximal effective inhibitory concentration; HaCat, human keratinocytes; NHDF, neonatal human dermal fibroblasts; SD, standard deviation.

FIG. 2 describes data demonstrating Compound 1 is a potent inhibitor of HSV-1 and HSV-2 clinical isolates. The value “n” denotes the number of clinical isolates tested. Each point represents an individual isolate and the horizontal line depicts the mean EC₅₀ across all clinical isolates tested. EC₅₀, half-maximal effective inhibitory concentration.

FIG. 3A describes data demonstrating Compound 1 has a high barrier to resistance in vitro. In particular, it shows the dose-escalation summary for HSV-1 and HSV-2.

FIG. 3B describes data demonstrating Compound 1 has a high barrier to resistance in vitro. In particular, it shows the genotyping of HSV-1 and HSV-2 from resistance selection. In Panel A, frequencies of variants detected in ABI-Compound 1—and pritelivir-treated cultures (≥15%) are indicated in parentheses. In Panel B, variant frequency (%) is compared with unpassaged virus. DMSO, dimethyl sulfoxide; EC₅₀, half-maximal effective inhibitory concentration.

FIG. 4 describes data demonstrating Compound 1 is more resilient to binding site variations than pritelivir.

FIG. 5 describes data demonstrating Compound 1 has a favorable oral pharmacokinetic profile in preclinical species, including rodent, primate, and canine, which supports once-weekly oral dosing. As shown, in vivo systemic clearance is lower than in vitro predicted clearance in nonclinical species.

FIG. 6 describes data demonstrating rat and human plasma protein binding for Compound 1. Compound 1 shows a similar unbound fraction between rat and human plasma; therefore, the restriction factor observed in rats is used for human PK projections. FIGS. 5 and 6 support a once-weekly 250-mg dose of Compound 1 is projected to achieve efficacious coverage in humans. Data are mean±SD for plasma concentration, % free in plasma, and in vitro clearance; mean for in vivo clearance. Parentheses indicate projected value. Plasma free fraction data were generated with Dianorm equilibrium dialysis device. Cyno, cynomolgus monkey; restriction factor, ratio of the in vitro predicted clearance to the observed in vivo clearance; SD, standard deviation.

FIG. 7A describes data demonstrating that Compound 1 reduces the number of HSV lesions in a Guinea Pig Model of Recurrent HSV Infection. In particular, it describes an in vivo efficacy time course.

FIG. 7B describes data demonstrating that Compound 1 reduces the number of HSV lesions in a Guinea Pig Model of Recurrent HSV Infection. In particular, it describes data demonstrating that cumulative mean lesion score of Compound 1 in the Guinea Pig Model. Compound 1 significantly reduces the development of recurrent lesions, after latency is established on day 14, in guinea pigs treated continuously with formulated chow at therapeutically relevant concentrations. PK sampling at 21, 49, 77, and 105 days post-infection. Compound 1 (0.04% weight/weight) plasma concentrations remain 8-fold greater than the guinea pig protein-adjusted EC₉₅ (133 nM). EC₉₅, 95% effective inhibitory concentration.

### DETAILED DESCRIPTION OF THE INVENTION

The present disclosure relates to novel uses of Compound 1:

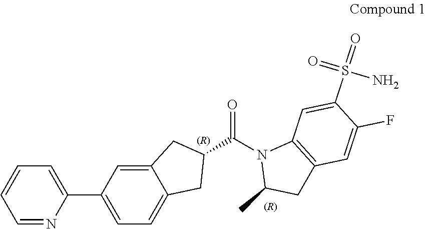

for the treatment of a viral infection unresponsive to treatment with one or more first antiviral therapy. Compound 1 was previously disclosed in WO2023225162, which relates to indolinyl compounds that inhibit viral helicase-primase; the use of the compounds for the preparation a medicament for the treatment of diseases and/or condition through inhibiting viral helicase-primase; use of those compounds in the treatment of viral infections; and intermediates for its preparation and to pharmaceutical compositions containing those compounds.

### Definitions

Unless otherwise defined, all technical and scientific terms used herein have the same meaning as commonly understood by one of ordinary skill in the art. In case of conflict, the present document, including definitions, will control. Methods and materials are described below, although methods and materials similar or equivalent to those described herein may be used in practice or testing of the present disclosure. All publications, patent applications, patents and other references mentioned herein are incorporated by reference in their entirety. The materials, methods, and examples disclosed herein are illustrative only and not intended to be limiting.

As used herein, the articles “a,” “an,” and “the” are used herein to refer to one or to more than one (i.e., to at least one) of the grammatical object of the article. By way of example, “an element” can mean one element or more than one element.

In this application, the use of “or” means “and/or” unless stated otherwise. The terms “and/or” and “any combination thereof” and their grammatical equivalents as used herein, may be used interchangeably. These terms may convey that any combination is specifically contemplated. Solely for illustrative purposes, the following phrases “A, B, and/or C” or “A, B, C, or any combination thereof” may mean “A individually; B individually; C individually; A and B; B and C; A and C; and A, B, and C.” The term “or” may be used conjunctively or disjunctively, unless the context specifically refers to a disjunctive use.

Furthermore, use of the term “including” as well as other forms, such as “include,” “includes,” and “included,” is not limiting.

Reference in the specification to “some embodiments,” “an embodiment,” “one embodiment” or “other embodiments” or “some aspects”, “an aspect” or “one aspect” means that a particular feature, structure, or characteristic described in connection with the embodiment and/or aspect is included in at least some embodiments and/or aspects, but not necessarily all, of the present disclosure.

As used in this specification and the claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification may be implemented with respect to any method or composition of the disclosure, and vice versa. Furthermore, compositions of the present disclosure may be used to achieve methods of the present disclosure.

As used herein, the term “about” or “approximately” refers to a quantity, level, value, number, frequency, percentage, dimension, size, amount, weight or length that varies by as much as 10, 9, 8, 7, 6, 5, 4, 3, 2 or 1% to a reference quantity, level, value, number, frequency, percentage, dimension, size, amount, weight or length. In some embodiments, the terms “about” or “approximately” when preceding a numerical value indicates the value plus or minus a range of 10%, 5%, or 1%.

The terms “Individual,” “patient,” or “subject” are used interchangeably and include any animal, including mammals, preferably mice, rats, other rodents, rabbits, dogs, cats, swine, cattle, sheep, horses, or primates, and most preferably humans. The compounds or pharmaceutical compositions of the disclosure may be administered to a mammal, such as a human, but may also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, dogs, primates, and the like). The mammal treated in the methods of the disclosure is desirably a mammal in which treatment of HSV infection is desired.

The term “Pharmaceutically acceptable” includes molecular entities and compositions that do not produce an adverse, allergic, or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, fillers, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable excipients.

The term “pharmaceutically acceptable salt(s)” as used herein refers to salts of acidic or basic groups that may be present in compounds used in the compositions. Compounds included in the present compositions that are basic in nature may form a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups: for example, one amino and one carboxylic acid group. In such a case, the compound may exist as an acid addition salt, a zwitterion, or a base salt.

The term “therapeutically effective amount” or “effective amount” as used herein refers to the amount of the subject compound that will elicit the biological or medical response of a tissue, system or animal, (e.g., mammal or human) that is being sought by the researcher, veterinarian, medical doctor or other clinician. The compounds or pharmaceutical compositions of the disclosure are administered in therapeutically effective amounts to treat a disease.

Alternatively, a therapeutically effective amount of a compound is the quantity required to achieve a desired therapeutic and/or prophylactic effect.

The term “treating” includes any effect, e.g., lessening, reducing, modulating, providing a protective effect, or eliminating, a viral infection, that results in the improvement of the disease.

The compounds disclosed herein may exist in solvated as well as unsolvated forms with pharmaceutically acceptable solvents such as water, ethanol, and the like, and it is intended that the disclosure embrace both solvated and unsolvated forms. In one embodiment, the compound is amorphous. In one embodiment, the compound is a single polymorph. In another embodiment, the compound is a mixture of polymorphs. In another embodiment, the compound is in a crystalline form.

The disclosure also embraces isotopically labeled compounds of the disclosure, which are identical to those recited herein, except that one or more atoms are replaced by an atom having an atomic mass or mass number different from the atomic mass or mass number usually found in nature. Examples of isotopes that may be incorporated into compounds of the disclosure include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium.

Certain isotopically labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the disclosure may generally be prepared by following procedures analogous to those disclosed in the examples herein by substituting an isotopically labeled reagent for a non-isotopically labeled reagent.

### Methods of Use:

Thus, one embodiment of the present disclosure provides a method for the treatment of a HSV infection unresponsive to treatment with one or more first antiviral therapy in a subject in need thereof, comprising: administering to the subject a therapeutically effective amount of Compound 1:

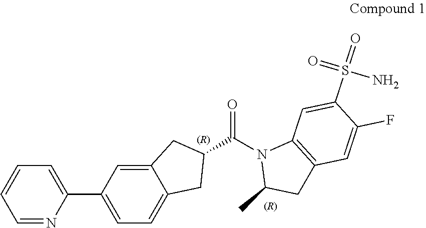

or a pharmaceutically acceptable salt thereof.

In another aspect, the disclosure provides a method for the treatment of a first viral infection unresponsive to treatment with one or more antiviral therapy in a subject in need thereof, comprising administering to the subject a pharmaceutical composition comprising a therapeutically effective amount of Compound 1, or a pharmaceutically acceptable salt thereof, and a pharmaceutically acceptable excipient.

The term “unresponsive to treatment” as used herein refers to a response from administration of an antiviral therapy, or a combination of therapies, that does not result in an therapeutically effective reduction of viral load, results in no decrease in the time of infection without therapeutically effective reduction of viral load, results in a diminished reduction of viral load or reduced potency as compared to the one or more compounds administered and described herein, or does not result in an improvement of symptoms.

An unresponsive response may be due to a variety of underlying mechanisms, including drug resistance. For example, in the case of HSV antiviral therapy, the HSV may become resistant to acyclovir due to mutations resulting from exposure to the drug. Thus, in one aspect, the infection is resistant to acyclovir. In another aspect, HSV comprises one or more mutations resulting from exposure to acyclovir. In other aspects, HSV may become resistant to a pritelivir due to mutations in the helicase-primer gene. In one aspect, the infection is resistant to pritelivir. In another aspect, the HSV comprises one or more mutations in the UL5 gene. In one aspect, the mutations are UL5-K355 mutations. In one aspect, the mutation is a K356N, K355N, K355T, K355R, L805I, S497N or S498N mutation in the UL5 gene. In one aspect, the mutations is a K355R with a L805I mutation, or a K355N with a S497N mutation, each double mutation in the UL5 gene. In one aspect, the HSV comprises one or more mutations in the UL52 gene. In one aspect, the mutation is a A906V mutation in the UL52 gene. In some aspects, the mutation is a K355R with L805I mutation in the UL5 gene, and a A906V mutation in the UL52 gene. Other mutations and combination of mutations within genes and between genes are contemplated herein. Thus, in one aspect described herein, the infection may be resistant to treatment with one or more antiviral therapy.

Alternatively, unresponsive to a treatment may also be the result of varying potency. Thus, while a first antiviral therapy, or combination, may have a first potency that is less than a subsequent therapy, which subsequent therapy is considered more potent.

As noted in the Examples, Compound 1 of the present disclosure displays enhanced antiviral activity against HSV as compared to other antiviral therapies. Thus, one aspect described herein, is a method of treating an HSV infection previously treated with acyclovir or pritelivir, comprising administration of Compound 1. In some aspects, administration of Compound 1 may result in about a 2 fold, about a 4 fold, about a 10 fold, about a 20 fold, about a 30 fold, about a 40 fold, about a 50 fold, about a 60 fold, about a 70 fold, about a 80 fold, about a 90 fold, a about 100 fold, about a 200 fold, about a 300 fold, about a 400 fold, about a 500 fold, about a 600 fold, about a 700 fold, about a 800 fold, about a 900 fold, about a 1,000 fold, about a 2,000 fold, about a 3,000 fold, about a 4,000 fold, about a 5,000 fold, about a 6,000 fold, about a 7,000 fold, about a 8,000 fold, about a 9,000 fold, or about a 10,000 fold increase in potency of the first antiviral therapies. In one aspect, Compound 1 is about 2 fold to about 15 fold more potent than a first antiviral therapy, inclusive of intermittent values. In another aspect Compound 1 is about 1000 fold to about 2000 fold more potent than a first antiviral therapy, inclusive of intermittent values.

In one aspect, Compound 1 is about greater than 12 fold more potent than pritelivir. In another aspect, Compound 1 is about greater than 1500 fold more potent than acyclovir.

Thus, another embodiment described herein is a method for the treatment of HSV infection having reduced susceptibility to acyclovir, in a subject in need thereof, the method comprising: administering to the subject a therapeutically effective amount of Compound 1, or a pharmaceutically acceptable salt or solvate thereof:

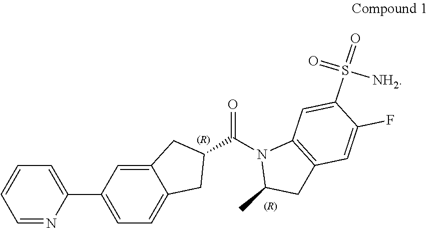

Another embodiment described herein is a method for the treatment of HSV infection having reduced susceptibility to pritelivir, in a subject in need thereof, the method comprising: administering to the subject a therapeutically effective amount of Compound 1:

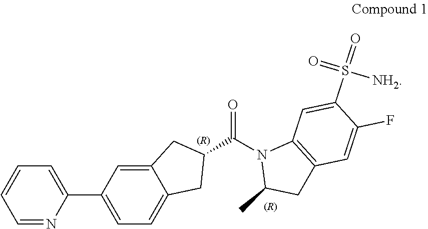

The term “reduced susceptibility” as used herein refers to a reduction in the effect of the administered drug.

Another embodiment described herein is a method of treatment of an HSV infection previously treated with an antiviral therapy in a subject in need thereof, the method comprising: administering to the subject a therapeutically effective amount of Compound 1:

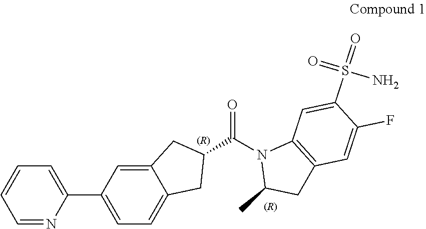

or a pharmaceutically acceptable salt thereof; wherein the HSV virus is not a wild-type virus or wherein the HSV virus is mutated as a result of exposure to a first antiviral therapy.

In some aspects, the infection may be a viral infection. In some aspects, the virus may be any virus, including but not limited to Respiratory Syncytial virus (RSV), Herpes Simplex virus (HSV), Influenza (Flu), Rhinovirus, Adenovirus, Parainfluenza, Coxackie, or Coronavirus.

In one aspect, the infection is an HSV infection. HSV infections may include, but are not limited to, three subfamilies (i.e., a, p and y) based upon their biological characteristics and the family consists of eight members, i.e., Herpes Simplex Virus subtype type 1 and 2 (HSV-1, HSV-2), Varicella Zoster Virus (VZV), Epstein-Barr virus (EBV), Cytomegalovirus (CMV), and human herpes viruses 6-8 (HHV 6-8). In one aspect, the infection is an HSV-1 infection. In another aspect, the infection is an HSV-2 infection.

In another aspect, the HSV virus comprises one or more mutations. In another aspect, the HSV comprises one or more mutations in the UL5 gene. In one aspect, the mutations are UL5-K355 mutations. In one aspect, the mutation is a K356N, K355N, K355T, K355R, L805I, S497N or S498N mutation in the UL5 gene. In one aspect, the mutations is a K355R with a L805I mutation, or a K355N with a S497N mutation, each double mutation in the UL5 gene. In another aspect, the HSV comprises one or more mutations in the UL52 gene. In one aspect, the mutation is a A906V mutation in the UL52 gene. In some aspects, the mutation is a K355R with a L805I mutation in the UL5 gene, and a A906V mutation in the UL52 gene. Other mutations and combination of mutations within genes and between genes are contemplated herein.

In some aspects, the subject is a herpes-positive patient. In other aspects, the subject is a herpes-simplex-positive patient. In some aspects, the infection is a Herpes simplex infection and the subject displays symptoms such as Herpes labialis, Herpes genitalis, HSV-related keratitis, encephalitis, or pneumonia. In another aspect, the infection is a Herpes simplex infection and the subject displays symptoms such as suppressed immune system (for example AIDS patients, cancer patients, patients having a genetic immunodeficiency, transplant patients). In another aspect, the infection is a Herpes simplex infection, and the subject is a new-born child or infant.

### Combination Therapies

The compounds according to the present disclosure are also useful for the treatment of a viral infection, in particular where the viral infection is a Herpes simplex viral infection, which is unresponsive to treatment with one or more first antiviral therapy, in combination with other active ingredients.

In one aspect, the present disclosure provides a method for the treatment of a viral infection unresponsive to treatment with one or more first antiviral therapy, and in particularly in a subject in need thereof, the method comprising: administering to the subject a therapeutically effective amount of Compound 1, or a pharmaceutically acceptable salt thereof, in combination with an antiviral therapy, including in combination with the first antiviral therapy, despite the infection being unresponsive to treatment with the first antiviral therapy. Thus one aspect of the present method comprises administering Compound 1, or a pharmaceutically acceptable salt thereof, in combination with the first antiviral therapy.

In some aspects, the antiviral therapy is selected from the group consisting of acyclovir, penciclovir, famciclovir, ganciclovir and valacyclovir, foscarnet trifluridine, pritelivir or amenamevir. In some aspects, the first antiviral therapy is selected from the group consisting of acyclovir, penciclovir, famciclovir, ganciclovir and valacyclovir, foscarnet, trifluridine, pritelivir or amenamevir.

Even when an HSV virus is resistant to either acyclovir or pritelivir alone, administration of either compound in combination with Compound 1 may be effective. Thus, in one aspect, Compound 1 may be administered with acyclovir, where acyclovir is considered the first antiviral therapy. In yet another aspect, Compound 1 may be administered with pritelivir, where pritelivir is considered the first antiviral therapy.

### Formulations and Administration

Compound 1 of the present disclosure may be converted in a known manner into the customary formulations, such as tablets, sugar-coated tablets, pills, granules, aerosols, syrups, emulsions, suspensions, and solutions, using inert, nontoxic, pharmaceutically suitable carriers and solvents. Here, the therapeutically active compound should in each case be present in a concentration of about 0.5 to 90% by weight of the total mixture, i.e., in amounts which are sufficient to achieve the dosage range indicated.

The formulations are prepared, for example, by extending the active compounds with solvents and/or excipients, if appropriate using emulsifiers and/or dispersants, it being possible, for example, if the diluent used is water, to use, if appropriate, organic solvents as auxiliary solvents.

Administration is carried out in a customary manner, including orally, parenterally, topically, prelingually or intravenously.

In the case of parenteral administration, solutions or suspensions of the active compounds using suitable liquid carrier and excipients may be employed.

In general, it has proved advantageous in the case of intravenous administration to administer amounts of from approximately 0.001 to 20 mg/kg, preferably approximately 0.01 to 10 mg/kg, of bodyweight to achieve effective results, and in the case of oral administration the dose is approximately 0.01 to 30 mg/kg, preferably 0.1 to 20 mg/kg, of bodyweight.

In some instances, it may be necessary to depart from the amounts mentioned, namely depending on the bodyweight or on the type of administration route, on the individual response to the medicament, the manner of its formulation and the time or interval at which administration takes place. Thus, in some cases it may be adequate to manage with less than the abovementioned minimum amount, while in other cases the upper limit mentioned must be exceeded. In the case of the administration of relatively large amounts, it may be advisable to divide this into several individual administrations over the course of the day.

If appropriate, it may be useful to combine the compounds according to the invention with other active substances, in particular antiviral active substances.

The compounds used in the present disclosure may be in the form of a pharmaceutically acceptable salt, cocrystal or a solvate. The term “pharmaceutically acceptable salts” refers to salts prepared from pharmaceutically acceptable non-toxic bases or acids, including inorganic bases or acids and organic bases or acids. In case the compounds of the present invention contain one or more acidic or basic groups, the invention also comprises their corresponding pharmaceutically or toxicologically acceptable salts, in particular their pharmaceutically utilizable salts. Thus, the compounds of the present invention which contain acidic groups may be used according to the invention, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine or amino acids. The compounds of the present invention which contain one or more basic groups, i.e., groups which may be protonated, may be used according to the invention in the form of their addition salts with inorganic or organic acids. Examples of suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the present invention simultaneously contain acidic and basic groups in the molecule, the invention also includes, in addition to the salt forms mentioned, inner salts or betaines (zwitterions). The respective salts may be obtained by customary methods which are known to the person skilled in the art like, for example, by contacting these with an organic or inorganic acid or base in a solvent or dispersant, or by anion exchange or cation exchange with other salts. The present invention also includes all salts of the compounds of the present invention which, owing to low physiological compatibility, are not directly suitable for use in pharmaceuticals but which may be used, for example, as intermediates for chemical reactions or for the preparation of pharmaceutically acceptable salts.

In practical use, Compound 1 may be combined as the active ingredient in intimate admixture with a pharmaceutical carrier according to conventional pharmaceutical compounding techniques. The carrier may take a wide variety of forms depending on the form of preparation desired for administration, e.g., oral, or parenteral {including intravenous). In preparing the compositions for oral dosage form, any of the usual pharmaceutical media may be employed, such as, for example, water, glycols, oils, alcohols, flavouring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or non-aqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds may also be administered intranasally as, for example, liquid drops or spray or as eye drops.

The tablets, pills, capsules, and the like may also contain a binder such as hydroxypropyl methylcellulose, or polyvinylpyrrolidone; diluent or fillers such as microcrystalline cellulose, dicalcium phosphate, lactose, or mannitol; a disintegrating agent such as croscarmellose sodium, polyvinylpyrrolidone, or sodium starch glycolate; a lubricant such as magnesium stearate or sodium stearyl fumarate; a glidant such as silicon dioxide; and a sweetening agent such as sucrose or saccharin. When a dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compound 1 may also be administered parenterally. Solutions or suspensions may be prepared in water suitably mixed with a surfactant such as hydroxypropyl cellulose, sodium lauryl sulfate, or polysorbate. Dispersions may also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier may be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present invention. For example, oral, rectal, topical, parenteral (including intravenous), ocular, pulmonary, nasal, and the like may be employed. Dosage forms include tablets, troches, dispersions, suspensions, solutions, capsules, creams, ointments, aerosols, and the like. Compounds of the present invention may be administered orally or as eye drop. The compounds of the present invention may also be administered orally. The effective dosage of active ingredient employed may vary depending on the particular compound employed, the mode of administration, the condition being treated, and the severity of the condition being treated. Such dosage may be ascertained readily by a person skilled in the art.

Compound 1 may also be present in combination with additional active ingredients, in particular, with one or more active ingredients exhibiting advantageous effects in the treatment of any of the disorders or diseases as described herein. Compound 1 may be present in a composition in combination with at least one further active substance being effective in treating a disease or disorder associated with viral infections (antiviral active compounds), preferably a disease or disorder being associated with viral infections caused by herpes viruses, such as in particular by Herpes simplex viruses (i.e., combination therapy). The at least one further active substance being effective in treating a disease or disorder associated with viral infections (antiviral active compounds) are preferably selected from the group consisting of nucleosidic drugs such as acyclovir, valacyclovir, penciclovir, ganciclovir, famciclovir and trifluridine, as well as compounds such as foscarnet and cidofovir.

Accordingly, the present invention further relates to a pharmaceutical composition comprising Compound 1 and at least one pharmaceutically acceptable carrier and/or excipient and/or at least one further active substance being effective in treating a disease or disorder associated with viral infections (antiviral active compounds).

Compound 1 may be converted in a known manner into customary formulations, such as tablets, caplets, sugar-coated tablets, pills, granules, aerosols, syrups, pharmaceutically suitable carriers, and solvents. Here, the therapeutically active compound should in each case be present in a concentration of about 0.1 to 90% by weight of the total mixture, i.e., in amounts which are sufficient to achieve the dosage range indicated.

The formulations are prepared, for example, by extending the active compounds with solvents and/or excipients, if appropriate using emulsifiers and/or dispersants, if being possible, for example, if the diluent used is water, to use, if appropriate, organic solvents as auxiliary solvents.

Administration is carried out in a customary manner, preferably orally, parenterally or topically, in particular perlingually or intravenously.

In the case of parenteral administration, solutions or suspensions of the active compounds using suitable liquid carrier materials may be employed.

In general, it has proved advantageous in the case or intravenous administration to administer amounts of from approx. 0.001 to 20 mg/kg, preferably approx. 0.01 to 10 mg/kg of bodyweight to achieve effective results, and in the case of oral administration the dose is approx. 0.01 to 30 mg/kg, preferably 0.1 to 20 mg/kg of body weight.

Despite this, it may be necessary, if appropriate, to depart from the amounts mentioned, namely depending on the bodyweight or on the type of the administration route, on the individual response to the medicament, the manner of its formulation and the time or interval at which administration takes place. Thus, in some cases it may be adequate to manage with less than the abovementioned minimum amount, while in other cases the upper limit mentioned must be exceeded. In the case of administration of relatively large amounts it may be advisable to divide this into several individual administrations over the course of the day.

The features and other details of the disclosure will now be more particularly described. Before further description of the present disclosure, certain terms employed in the specification, examples and appended claims are collected here. These definitions should be read in view of the remainder of the disclosure and as understood by a person of skill in the art. Unless defined otherwise, all technical and scientific terms used herein have the same meaning as commonly understood by a person of ordinary skill in the art.

### EXAMPLES

### Preparation of Compound 1

Compound 1 can be prepared in several ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

At least some of the compounds identified as “intermediates” herein are contemplated as compounds of the disclosure.

### Synthesis of (R)-5-fluoro-2-methyl-1-((R)-5-(pyridin-2-yl)-2,3-dihydro-1H-indene-2-carbonyl)indoline-6-sulfonamide (Compound 1)

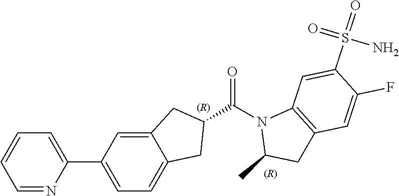

Compound 1 was prepared according to the procedures described in Example 35 of PCT Application No. PCT/US2023/022679.

### Preparation of Intermediate 2: 5-(pyridin-2-yl)-2,3-dihydro-1H-indene-2-carboxylic Acid

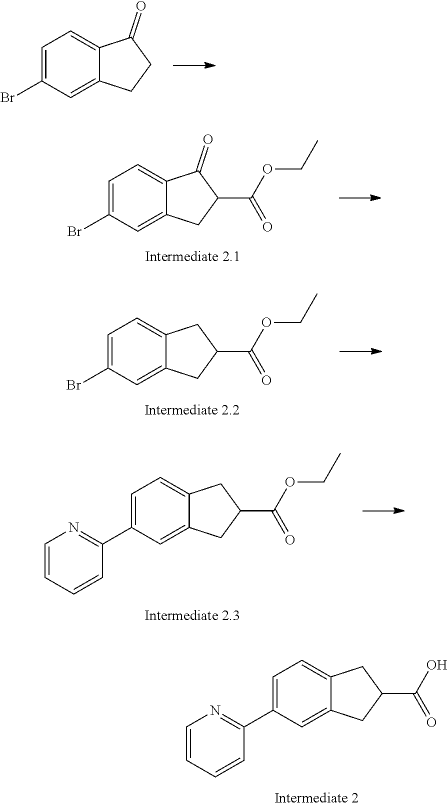 [SMILES: O=C1CCc2cc(Br)ccc21 ; CCOC(=O)C1Cc2cc(Br)ccc2C1=O ; CCOC(=O)C1Cc2ccc(Br)cc2C1 ; CCOC(=O)C1Cc2ccc(-c3ccccn3)cc2C1 ; O=C(O)C1Cc2ccc(-c3ccccn3)cc2C1]

Preparation of Intermediate 2.1: To a stirred suspension of 60% NaH (22.72 g, 2 equiv) in THF (1000 mL) at 0° C. was added diethyl carbonate (86 mL, 1.5 equiv). To this was added 5-bromo-indan-1-one (100 g, 473.1 mmol) portion wise at this temperature. The reaction mixture continued to stir at this temperature until gas evolution subsided. The reaction mixture was slowly heated to 50° C. and maintained stirring for 2 h. Progress of the reaction was monitored by TLC, and after consumption of starting material, the reaction mixture was allowed to cool to RT. The reaction was diluted with EtOAc (10V). To this was added 3N aq HCl dropwise. Both layers were separated. Aqueous layer was extracted with EtOAc (2×5V). The combined organic layer was washed with brine solution (5V), dried (Na2SO4) and evaporated to obtain the crude, which was purified by column chromatography on silica gel to afford Intermediate 2.1. LCMS: 283.0 [M+H].

Preparation of Intermediate 2.2: To a solution of Intermediate 2.1 (56 g, 197.9 mmol) in TFA (280 mL) at 0° C. was added triethyl silane (224 mL) dropwise and the reaction mixture was stirred at rt for 24 h. Progress of the reaction was monitored by TLC. After consumption of starting materials, the reaction mixture was evaporated to dryness under reduced pressure to obtain the crude. The crude was purified by column chromatography on silica gel to obtain Intermediate 2.2. LC/MS: 269.0 [M+H].

Preparation of Intermediate 2.3: A stirred solution of Intermediate 2.2 (40 g, 148.7 mmol) and 2-tributylstannylpyridine (57.5 g, 1.05 equiv) in 1,4 dioxane (400 mL) was degassed for 10 min using argon, at which time Pd(PPh3)4 (8.6 g, 5 mol %) was added and again degassed for another 10 min. The reaction mixture was heated to 90° C. for 16 h. The reaction mixture was filtered through Celite pad and the Celite pad was washed with ethyl acetate twice. The combined filtrate was evaporated to dryness to obtain crude. The crude was purified by column chromatography on silica gel to afford Intermediate 2.3. LC/MS: 268.1 [M+H].

Preparation of Intermediate 2: To a stirred solution of Intermediate 2.3 (35 g, 131.1 mmol) in methanol (350 mL) at 0° C. was added 2N aq NaOH solution (140 mL, 2.2 equiv) and the reaction mixture was stirred at rt for 4 h. After completion of the reaction, the reaction mixture was concentrated under reduced pressure to 100 mL. The residue was diluted with water (10V) and the aqueous layer washed with ethyl acetate (2×2V). The aqueous phase was neutralized with 2N aq HCl solution and extracted with 10% Methanol/DCM (3×5V). The organic layer was dried over sodium sulfate and concentrated to afford the crude. Then the crude was taken with 10% isopropanol/toluene (10V) solution and treated with activated carbon. The solvent was then removed under reduced pressure. The residue was taken in 30% toluene/hexane, stirred for 30 min and filtered, and then dried under vacuum. This process was repeated two additional times to afford Intermediate 2. LC/MS: 238.1 [M−H].

### Preparation of Intermediate 19: (R)-5-(pyridin-2-yl)-2,3-dihydro-1H-indene-2-carboxylic Acid

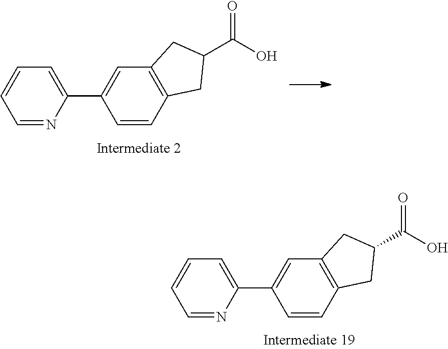 [SMILES: O=C(O)C1Cc2ccc(-c3ccccn3)cc2C1 ; O=C(O)[C@@H]1Cc2ccc(-c3ccccn3)cc2C1]

Intermediate 19 was obtained as the first eluent of SFC purification of Intermediate 2 using a Chiralpak AD-H column with 20% methanol as co-solvent. LC/MS: 238.1 [M−H].

### Preparation of Intermediate 21

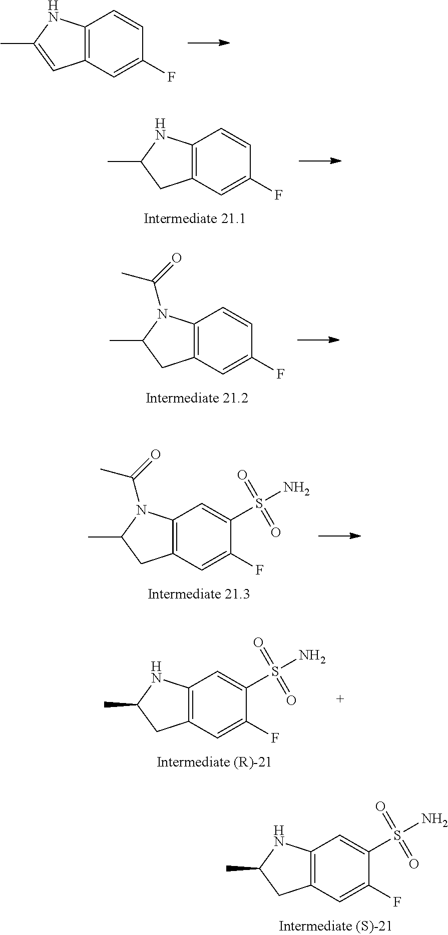 [SMILES: Cc1cc2cc(F)ccc2[nH]1 ; CC1Cc2cc(F)ccc2N1 ; CC(=O)N1c2ccc(F)cc2CC1C ; CC(=O)N1c2cc(S(N)(=O)=O)c(F)cc2CC1C ; C[C@@H]1Cc2cc(F)c(S(N)(=O)=O)cc2N1 ; C[C@@H]1Cc2cc(F)c(S(N)(=O)=O)cc2N1]

Preparation of Intermediate 21.1: 5-fluoro-2-methylindoline (40 g, 268 mmol, 1 eq) was taken up in acetic acid (200 mL, 5V). NaBH₃CN (50 g, 815 mmol, 3 eq) was added portion-wise while maintaining the temperature below 10° C. The resulting solution was warmed to RT and stirred for 3 h, at which time the reaction was diluted with ice-cold water (500 mL). The reaction was then extracted with EtOAc (3×100 mL). The combined organic layer was washed with brine (750 mL), dried over Na₂SO₄ and concentrated in vacuo. The crude Intermediate 21.1 was isolated as a thick yellow oil and used in the next step without further purification. LC/MS: 151.2 [M+H].

Preparation of Intermediate 21.2: To a stirred solution of Intermediate 21.1 (35 g, 86.6 mmol) in DCM (300 mL) was added triethylamine (35 mL, 1 vol) followed by acetyl chloride (35 mL, 1 vol) at 0° C. The reaction mixture was stirred at RT for 2 h. The residue was quenched with cold water (200 mL) and extracted with EtOAc (3×100 mL). The combined organic layer was washed with brine (750 mL), dried over Na₂SO₄ and concentrated in vacuo. The crude residue was purified by column chromatography (100-200 silica gel, eluted 8% EtOAc-Hexane) to afford Intermediate 21.2. LC/MS: 193.2 [M+H].

Preparation of Intermediate 21.3: To a stirred solution of Intermediate 21.2 (35 g, 0.011 mol) in chlorosulfonic acid (250 mL, 10 V) at 0° C. under nitrogen and the reaction mixture was stirred at 50° C. for 3 h. Upon completion of the reaction, the mixture was diluted with ice-cold water. The precipitate was filtered, taken up in dichloromethane (50 mL), and added to a solution of concentrated ammonium hydroxide (20 mL). After vigorous stirring for 15 minutes at RT, the solvent was removed under pressure and the resulting solid was filtered and rinsed with water to afford Intermediate 21.3. LC/MS: 272.3 [M+H].

Preparation of Intermediates (R)-21 and (S)-21: A racemic mixture of Intermediate 21.3 (30 g, 110 mmol, 1 equiv) in 500 mL of 2N sodium hydroxide was heated at 100° C. for 3 hours. The reaction was cooled to RT and the pH was adjusted to 7 with acetic acid. The precipitate was filtered, washed with water, and dried under vacuum. The resulting de-acetylated racemic mixture was then subjected to chiral SFC purification using a ChiralPak IG 240×4.6 mm column with a mobile phase consisting of 70:20:10 hexane:methanol:MTBE. Intermediate (R)-21 was collected as the second eluent (retention time 13.1 min). LC/MS: 230.1 [M+H]. Intermediate (S)-21 was collected as the first eluent (retention time 10.5 min). LC/MS: 230.1 [M+H].

### Preparation of Compound 1

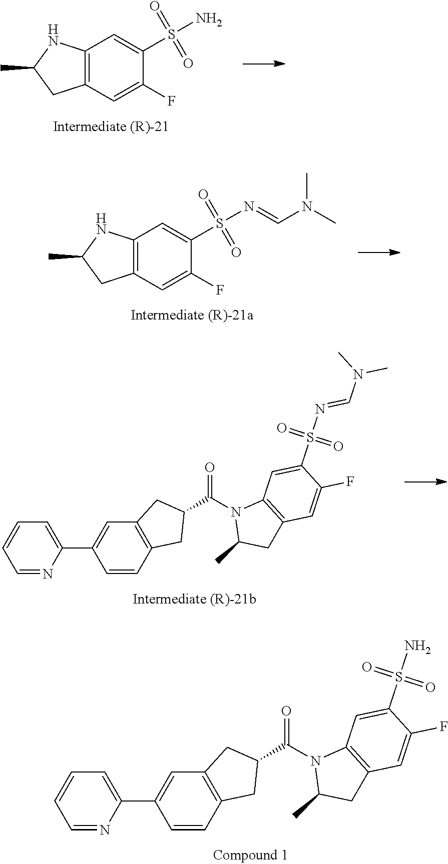

Intermediate (R)-21 was taken up in THF (10 V) and dimethylformamide dimethyl acetal (1.2 equiv) was added at once. The solution was stirred at RT for 30 minutes and the solvent was then removed under reduced pressure to afford the crude residue Intermediate (R)-21a (LC/MS: 286.1 [M+H]) which was used directly in the next step without further purification.

Intermediate (R)-21a was taken up in acetonitrile (10 V). Intermediate 19 (1 equiv) was added, followed by TCFH (2 equiv). The suspension was placed in a RT water bath and N-methylimidazole (5 equiv) was added dropwise. The solution stirred at RT for 1 hour, at which time LC/MS analysis indicated complete conversion to Intermediate (R)-21b (LC/MS: 507.2 [M+H]). Hydrazine hydrate (50 equiv) was then added at once and allowed to stir at RT for 30 minutes, after which time water was added (5 V). The resulting precipitate was filtered and dried under reduced pressure to afford Compound 1. ¹H NMR (400 MHz, DMSO-d6) b 8.69 (d, J=4.9 Hz, 1H), 8.54 (d, J=6.6 Hz, 1H), 8.10-7.84 (m, 4H), 7.58 (s, 2H), 7.51-7.24 (m, 3H), 4.90 (t, J=7.5 Hz, 1H), 3.77 (p, J=8.2 Hz, 1H), 3.46 (ddt, J=34.0, 17.5, 8.7 Hz, 3H), 3.20 (ddd, J=59.1, 16.3, 8.3 Hz, 2H), 2.80 (d, J=16.9 Hz, 1H), 1.30 (d, J=6.2 Hz, 3H). LC/MS: 452.1 [M+H].

### BIOLOGICAL ASSAY DATA

### Cells

Adult retinal pigment epithelial cells (ARPE-19, CRL-2302), neonatal primary normal human dermal fibroblasts (NHDFs, PCS-201-010), and Vero (CCL-81) cells were purchased from ATCC (Manassas, MD). Immortalized human keratinocytes (HaCaT, 300493) were purchased from Cytion (Sioux Falls, SD). Growth medium consisted of DMEM/F-12 with GlutaMAX (Thermo Fisher Scientific, Waltham, MA; 10565018) supplemented with 10% fetal bovine serum (FBS) (Thermo Fisher Scientific, 10082147) and Pen-Strep (Thermo Fisher Scientific; 15070063) for ARPE-19 cells or DMEM, high glucose, Glutamax (Thermo Fisher Scientific; 10569010) supplemented with 10% FBS and Pen-Strep for NHDF, HaCat, and Vero cells.

### Viruses

Inoculums of HSV-2 MS (VR-540) and HSV-1 KOS (VR-1493) strains were purchased from ATCC and propagated in Vero cells. Briefly, 2×10⁷ Vero cells were seeded in a T150 flask in growth medium, incubated for 1 day until 80-90% confluency was reached, and infected with HSV-1 or HSV-2 at multiplicity of infection (MOI) 0.01. When complete cytopathic effect (CPE) was observed (2-3 days), cells were scraped off and cell culture medium transferred in 50 mL conical vials to be spun at 1,000 rpm for 5 min to remove cell debris. Infectious supernatants were pooled, aliquoted, and stored at −80° C.

### HSV Mutant Generation

Single Mutant Generation—Six single mutants (UL52-A906V, UL5-K355N, UL5-K355R, UL5-K355T, UL5-L805I, and UL5-S497N) were generated in the mCherry bacmid system using en passant mutagenesis. As a template, wild-type pp28-sNLUC-encoding HSV-2 bacmid (Kropp), kindly provided by Dr. Viejo-Borbolla (Hannover Medical School), was used as a wildtype bacmid. Other wild-type bacmids may be used as a foundational template to create mutant forms. Briefly, a recombination template containing the mutation and Kan-resistance was amplified by PCR into the APHAI-I-Scel cassette and electroporated into GS1783 E. coli competent cells. KanR colonies were screened by sequencing to generate bacmid glycerol stocks and DNA was extracted using NucleoBond Xtra Bac purification kit (Takara Bio). Single mutant viruses were rescued by transfecting purified DNA into 293FT cells using TranslT-LT1 (Mirus Bio) and subsequently used to infect Vero cells for bulk virus generation. mCherry expression was used to validate and titer single mutant viruses.

Phenotyping Single Mutants—ARPE-19 cells were briefly trypsinized and bulk infected with HSV-2 single mutant virus at MOI 0.5 for one hour shaking at room temperature. ARPE-19 cells were seeded at a density of 25,000 cells/well in black clear bottom 96 well plates (Corning) and incubated at 37° C. for one hour. Treatment compounds (Compound 1 and ACV) were prepared and added to the plate in 2.5 to 4-fold dilution series and normalized to a final concentration of 0.5% DMSO. Cells were stained with Hoechst dye and mCherry signal was quantified using Cellomics (Thermo Fisher Scientific) for EC₅₀ determination 20 hours post infection. Uninfected controls were used to minimize background signal. Subsequently, cells were harvested using a PrepGEM DNA extraction kit (MicroGEM Int.) for qPCR validation following manufacturer's protocol. A cellular quantitative PCR-based assay was used to measure replication of HSV-1 and HSV-2 virus by quantifying UL30 (DNA polymerase) levels within the cell lysate. Briefly, a qPCR master mix (Quantifast Multiplex kit, Qiagen) was prepared with HSV UL30-FAM primer/probe mix (IDT) added to all samples, ran on a QuantStudio Real-time PCR machine (Qiagen) at 95° C. for 5 min, 40 cycles of 95° C. for 30 sec, and 60° C. for 30 sec. Quantitative HSV1/HSV2 genomic DNA (ATCC) used as a standard curve and relative gene expression was calculated via comparative Ct values (ΔΔCT).

### Antiviral Assay

Compounds were spotted onto 96-well assay plates (Corning, Corning, NY; 07-200-567) in 50 μL serum-free growth medium using a HP Digital Dispenser D300 (Hewlett Packard®, Palo Alto, CA) to create 10-point, 2.5-fold serial dilutions (four rows per compound). DMSO (Sigma, St. Louis, MO; D2650) was spotted onto remaining wells as vehicle control. Cells approaching confluence were dissociated using Tryp-LE (Thermo Fisher Scientific; A12177-01), resuspended in assay medium (growth medium with 2% FBS) and counted. Cells were bulk infected in a 50 mL conical vial rocking for 1 hour at room temperature with HSV-2 at a MOI of 0.05/0.1/0.05 (ARPE-19/NHDF/HaCaT) or HSV-1 at a MOI of 0.05 (ARPE-19) at 1×10⁶ cells/mL. Following infection, cells were spun down at 1,200 rpm for 7 minutes and resuspended in assay medium. 50 μL of infected cells (total cells: ARPE-19=25,000; NHDF=40,000; HaCaT=30,000) were added per well (on top of spotted compounds) for a total volume per well of 100 μL and a final concentration of DMSO of 0.5%. After incubating at 37° C. for 20 hours, cell culture medium was aspirated from all wells and replaced with 100 μL of prepGEM master mix (PrepGEM Tissue Kit, Zygem; PTI0500K). Plates were sealed with a breathable film (Millipore Sigma, Burlington, MA; A9224) and left on a plate shaker for 10 minutes. Lysates were then transferred onto a Microamp Optical 96-well PCR plate (Thermo Fisher Scientific; 4316813), placed in a thermocycler, and the following heat cycle was run: 10 minutes at 75° C., 5 minutes at 95° C., and hold at 4° C. Samples then either stored at −20° C. or analyzed by qPCR directly.

Quantification of Viral DNA Levels by qPCR

Lysates were brought to room temperature and a qPCR master mix was prepared using the QuantiNova Multiplex PCR kit (Qiagen, Germantown, MD; 208456). Briefly, qPCR reagents were combined to account for 5 μL of 4× Quantinove Master Mix, 0.1 μL of QN ROX reference dye, 1 μL of 20× primer-probe mix, and 8.9 μL of RNase-free water per well. The 20× primer-probe mix consisted of 10 μM of each primer (Forward: 5′-AGAGGGACATCCAGGACTTTGT-3′ (SEQ ID NO: 1), Reverse: 5′-CAGGCGCTTGTTGGTGTAC-3′ (SEQ ID NO: 2)) and 4 μM of the Taqman probe (5′-/56-FAM/AC CGC CGA A/ZEN/C TGA GCA/3IABkFQ/-3′ (SEQ ID NO: 3)). These were purchased from Integrated DNA Technologies (San Diego, CA) and target a conserved region of the HSV-1 and HSV-2 DNA polymerase gene (UL30). 15 μL of qPCR master mix and 5 μL of lysate were added to wells of a MicroAmp Optical ‘Fast’ PCR plate (Thermo Fisher Scientific; 4346906). Plates were sealed, spun for 1 minute at 1,200 rpm to remove bubbles, and placed in a QuantStudio 7 Flex real-time PCR system instrument (Applied Biosystems, Waltham, MA). Within the QuantStudio software, the 96-well ‘Fast’ plate format was selected, along with the Comparative Ct and Taqman chemistry modes. The following PCR 2-step program was then run: 2 minutes at 95° C. followed by 40 cycles of 30 seconds at 95° C. and 30 seconds at 60° C. qPCR Ct data was exported for downstream analysis.

### Biological Assays

### Example 1—HSV Laboratory Strains and Clinical Isolate Antiviral Assays

HSV-1 and HSV-2 laboratory strains were used to infect retinal epithelial (ARPE-19), human keratinocyte (HaCat), and neonatal human dermal fibroblast (NHDF) in the presence of compound. HSV-1 and HSV-2 clinical isolates were used to infect ARPE-19 in the presence of compound. Compound EC₅₀s were measured by qPCR using UL30 specific primers and reference HSV-1/HSV-2 genomic DNA.

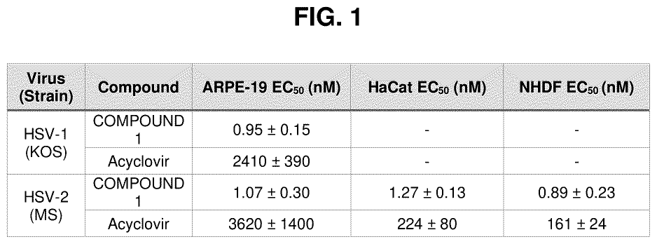

Compound 1 had potent antiviral activity against HSV-1 and HSV-2 replication in ARPE-19 cells. Similar potency was observed in other physiologically relevant cell lines (FIG. 1). Compound 1 was >2500-fold more potent than acyclovir in ARPE-19 cells against HSV-1 and HSV-2, and >150-fold more potent against HSV-2 in HaCat and NHDF cells.

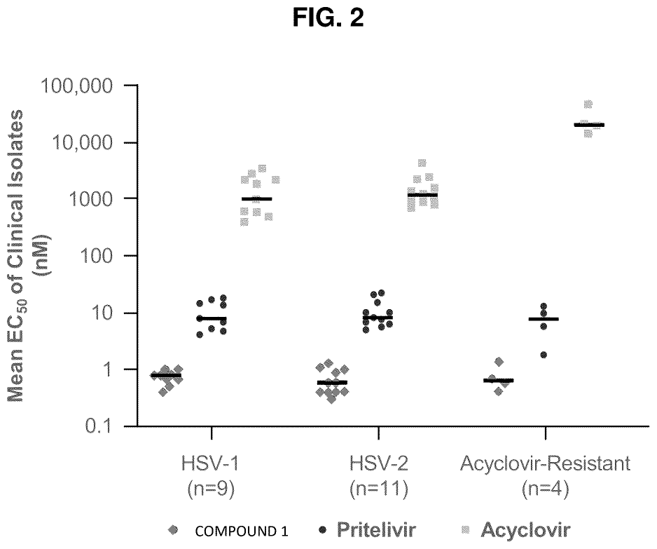

Furthermore, Compound 1 was active against HSV-1 and HSV-2 clinical isolates, including those with reduced susceptibility to acyclovir (FIG. 2). Compound 1 was >12-fold more potent than pritelivir against HSV-1 and HSV-2 clinical isolates and >1500-fold more potent than acyclovir and was found to remain active against all acyclovir-resistant HSV isolates tested

### Example 2—Viral Resistance Determination

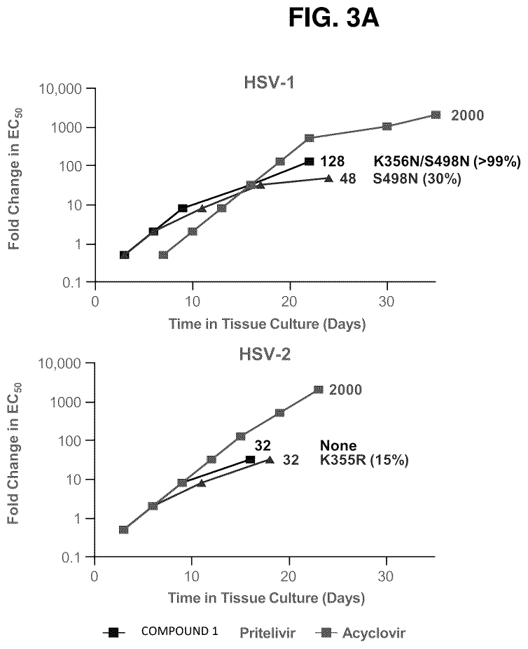

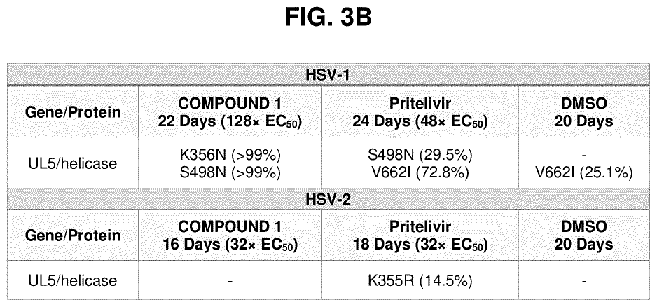

Vero cells infected with HSV-1 or HSV-2 were treated with escalating doses of compound until presence of full cytopathic effect (CPE). The cells and supernatant were processed for deep sequencing using gene-specific primers. FIG. 3A describes the dose-escalation summary for HSV-1 and HSV-2, where Compound 1 was found to have a higher barrier to resistance in vitro for both HSV-1 and HSV-2 populations passaged in Vero cells compared with acyclovir.

### Example 3—Genotype and Phenotypic Assessment of Resistant Mutations

A bacmid encoding HSV-2 MS strain with an mCherry reporter was used to generate mutant constructs via en passant mutagenesis. An image-based assay to measure cellular mCherry for EC₅₀ determination was used following infection of ARPE-19 cells with recombinant viruses. Phenotypic assessment of resistant mutations were evaluated by comparing their EC₅₀s with that of wild type virus.

FIG. 3B describes the results from genotyping HSV-1 and HSV-2 from the resistance selection. The K356N and S498N variants of HSV-1 UL5 are present at the highest Compound 1 passage concentration tested (128-fold ECso; FIG. 3B). For HSV-2, there were no variants in target genes UL5 and UL52 at the highest concentration of Compound 1 tested (32-fold EC₅₀) that produced CPE (FIG. 3B). Resistance selection data suggest that Compound 1 binds at the UL5/UL52 interface, consistent with Cryo-EM structure data (not shown).

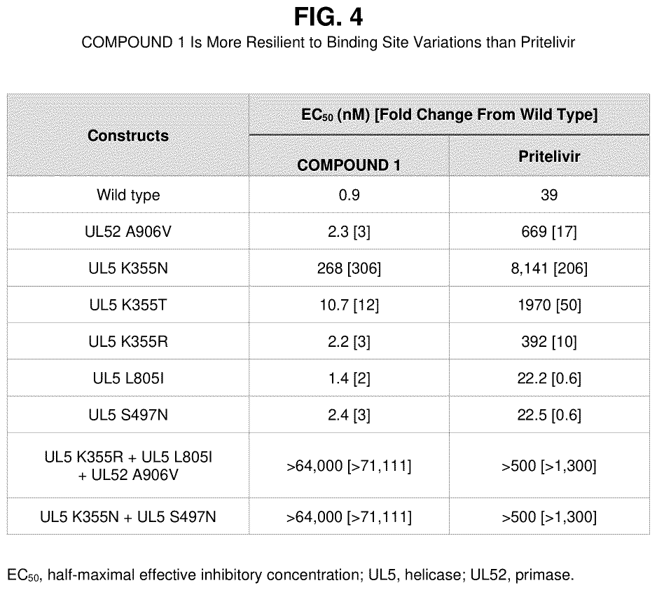

Furthermore, phenotypic assessment of UL5-K355 helicase variants, including those identified in vitro and in the clinic, reveals modest potency shifts for Compound 1 compared with pritelivir (FIG. 4). HSV-2 double and triple mutants display a high level of resistance against Compound 1 and pritelivir (FIG. 4).

### Reference Examples

### Pharmacokinetic Profiling

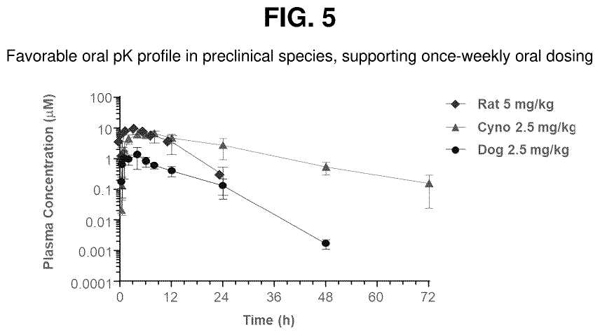

FIG. 5 describes data demonstrating Compound 1 has a favorable oral pharmacokinetic profile in preclinical species, including rodent, primate, and canine, which supports once-weekly oral dosing. As shown, in vivo systemic clearance is lower than in vitro predicted clearance in nonclinical species.

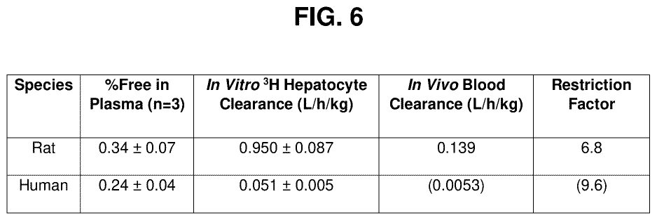

FIG. 6 describes data demonstrating rat and human plasma protein binding for Compound 1. Compound 1 shows a similar unbound fraction between rat and human plasma; therefore, the restriction factor observed in rats is used for human PK projections. FIGS. 5 and 6 support a once-weekly 250-mg dose of Compound 1 is projected to achieve efficacious coverage in humans.

### Guinea Pig Model of Recurrent HSV Infection

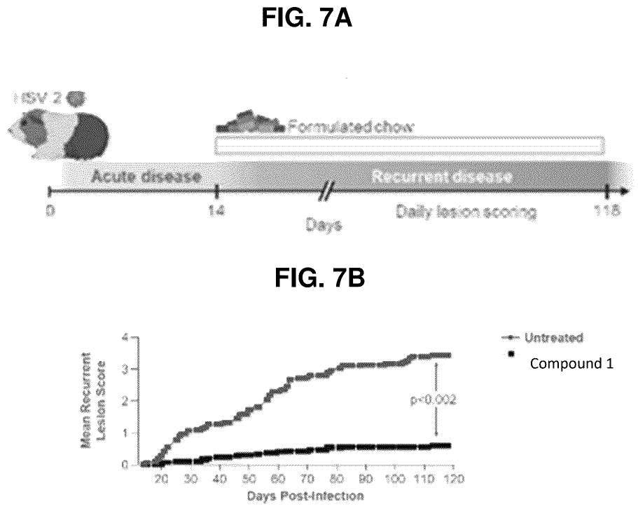

FIGS. 7A and 7B describe data demonstrating that Compound 1 reduces the number of HSV lesions in a Guinea Pig Model of Recurrent HSV Infection. FIG. 7A describes an in vivo efficacy time course. FIG. 7B describes data demonstrating that cumulative mean lesion score of Compound 1 in the Guinea Pig Model. Compound 1 significantly reduces the development of recurrent lesions, after latency is established on day 14, in guinea pigs treated continuously with formulated chow at therapeutically relevant concentrations.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The examples described herein are provided by way of illustration only and not by way of limitation. Those skilled in the art will readily recognize a variety of parameters that could be changed or modified to yield essentially similar results.

1. A method for the treatment of an HSV infection in a subject in need thereof, the method comprising:
administering to the subject a therapeutically effective amount of Compound 1: 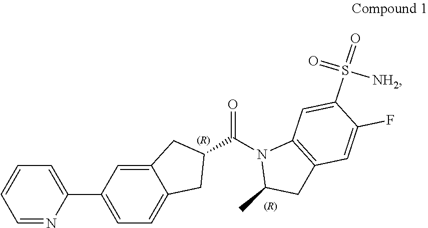
or a pharmaceutically acceptable salt thereof; wherein the virus is unresponsive to treatment with one or more first antiviral therapy.

2. The method of claim 1, wherein the first antiviral therapy is a nucleoside analogue or a helicase-primase inhibitor.

3. The method of claim 2, wherein the first antiviral therapy is a nucleoside analog, and the nucleoside analogue is acyclovir.

4. The method of claim 2, wherein the first antiviral therapy is a helicase-primase inhibitor, and the helicase-primase inhibitor is pritelivir.

5. The method of claim 1, wherein the infection is a Herpes Simplex Virus (HSV) infection.

6. The method of claim 5, wherein the HSV is an HSV-1 or HSV-2.

7. The method of claim 5, wherein the HSV comprises one or more mutations.

8. The method of claim 7, wherein the HSV comprises a A906V mutation in the UL52 gene.

9. The method of claim 1, wherein the infection is resistant to treatment with one or more antiviral therapy.

10. The method of claim 1, wherein Compound 1 is about greater than 1500 fold more potent than acyclovir.

11. The method of claim 1, wherein Compound 1 is about greater than 12 fold more potent than pritelivir.

12. A method for the treatment of a viral infection unresponsive to treatment with one or more first antiviral therapies in a subject in need thereof, the method comprising: administering to the subject a therapeutically effective amount of a pharmaceutical composition comprising Compound 1: 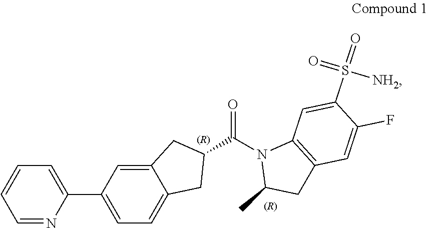
or a pharmaceutically acceptable salt thereof and a pharmaceutically acceptable excipient.

13. The method of claim 12, wherein the first antiviral therapy is a nucleoside analogue or a helicase-primase inhibitor.

14. The method of claim 13, wherein the nucleoside analogue is acyclovir.

15. The method of claim 13, wherein the helicase-primase inhibitor is pritelivir.

16. The method of claim 12, wherein the infection is an HSV infection.

17. The method of claim 16, wherein the HSV is an HSV-1 or HSV-2.

18. The method of claim 12, wherein the HSV is mutated.

19. The method of claim 18, wherein the HSV comprises a K355 mutation in the UL5 gene.

20. The method of claim 18, wherein the HSV comprises a K356N mutation in the UL5 gene.

21. The method of claim 18, wherein the HSV comprises a S498N mutation in the UL5 gene.

22. The method of claim 12, wherein the infection is resistant to treatment with one or more antiviral therapy.

23. The method of claim 12, wherein Compound 1 is about greater than 1500 fold more potent than acyclovir.

24. The method of claim 12, wherein Compound 1 is about greater than 12 fold more potent than pritelivir.

25. A method for the treatment of HSV infection previously treated with acyclovir, in a subject in need thereof, the method comprising: administering to the subject a therapeutically effective amount of Compound 1: 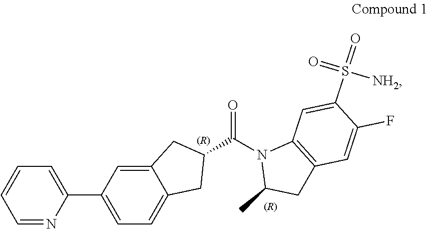
or a pharmaceutically acceptable salt thereof.

26. A method for the treatment of HSV infection previously treated with pritelivir, in a subject in need thereof, the method comprising: administering to the subject a therapeutically effective amount of Compound 1: 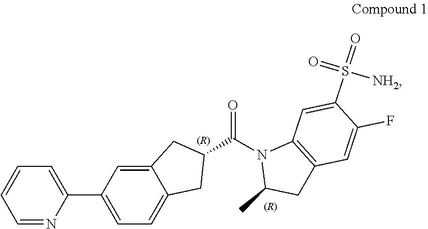
or a pharmaceutically acceptable salt thereof.

27. A method of treatment of an HSV infection previously treated with an antiviral therapy in a subject in need thereof, the method comprising:
administering to the subject a therapeutically effective amount of Compound 1: 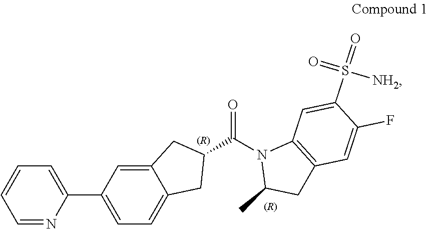
or a pharmaceutically acceptable salt thereof;
wherein the HSV virus comprises one or more mutations.

28. The method of claim 27, wherein the HSV comprises one or more mutations selected from the group consisting of K356N, K355N, K355T, K355R, L805I, S497N, and S498N mutation in the UL5 gene.

29. The method of claim 28, wherein the HSV comprises a A906V mutation in the UL52 gene.
